(19) United States
(12) Patent Application Publication
(10) Pub. No.: US 2019/0219582 A1
(43) Pub. Date: Jul. 18, 2019

(54) p16 EXPRESSION AND CANCER TREATMENT OUTCOME

(71) Applicant: NantOmics, LLC, Culver City, CA (US)

(72) Inventors: Fabiola Cecchi, Washington, DC (US); Todd Hembrough, Gaithersburg, MD (US); Yeoun Jin Kim, Gaithersburg, MD (US)

(21) Appl. No.: 16/251,409

(22) Filed: Jan. 18, 2019

Related U.S. Application Data
(60) Provisional application No. 62/618,897, filed on Jan. 18, 2018.

(51) Int. Cl. G01N 33/574; A61K 33/243; A61K 31/519

(52) U.S. Cl. CPC G01N 33/57419; A61K 31/519; A61K 33/243

(57) ABSTRACT

Methods are provided for identifying whether colorectal cancer (CRC) will be responsive to treatment with the combination of the therapeutic agents cisplatin and pemetrexed. Specified p16 fragment peptides are precisely detected and quantitated by SRM-mass spectrometry directly in formalin-fixed tissue sample that was obtained from the cancer patient and compared to a p16 reference level in order to determine if the CRC patient will positively respond to treatment with the combination of cisplatin and pemetrexed therapeutic agents.

### CROSS-REFERENCED APPLICATIONS

This application claims the benefit of U.S. Provisional Application No. 62/618,897 filed on 18 Jan. 2018, the entire contents of which are hereby incorporated by reference.

### FIELD

Methods are provided for treating cancer patients, for example colorectal cancer (CRC) patients, by assaying tumor tissue surgically-removed from patients and identifying those patients most likely to respond to treatment.

### BACKGROUND

Cisplatin, also known as cisplatinum, platamin, and neoplatin, is a member of a class of platinum-containing anti-cancer drugs, which also includes carboplatin and oxaliplatin. Once inside the cancer cell these platinum therapeutic agents bind to and cause crosslinking of DNA which damages DNA ultimately triggering apoptosis (programmed cell death) and death to cancer cells. Nucleotide excision repair (NER) is the primary DNA repair mechanism that removes the therapeutic platinum-DNA adducts from the tumor cell DNA.

Pemetrexed, also known as Alimta, is chemically similar to folic acid and is in the class of chemotherapy drugs called folate antimetabolites. It works by inhibiting three enzymes used in purine and pyrimidine synthesis: thymidylate synthase (TS); dihydrofolate reductase (DHFR); and glycinamide ribonucleotide formyltransferase (GARFT). By inhibiting the formation of precursor purine and pyrimidine nucleotides, pemetrexed prevents the formation of DNA and RNA, which are required for the growth and survival of both normal cells and cancer cells.

Cyclin-dependent kinase inhibitor 2A, also known as p16 and as multiple tumor suppressor 1, is a tumor suppressor protein that plays an important role in cell cycle regulation. US 2018/0177825 to Hembrough et al. reports the value of p16 as a predictor of lung cancer response to cisplatin/pemetrexed. The p16 functions by decelerating cell progression from G1 phase to S phase, and therefore acts as a tumor suppressor that is implicated in the prevention of cancers, notably melanoma, oropharyngeal squamous cell carcinoma, cervical cancer, and esophageal cancer. The p16 gene is frequently mutated or deleted in a wide variety of tumors. Expression of p16 and its involvement through the ROS pathway, chemotherapy-induced DNA damage, and/or cellular senescence leads to the buildup of p16 in cells and is implicated in aging of cells. The mechanism by which the combination of cisplatin and pemetrexed relates to expression and/or function of the p16 protein in cancer cells is unknown.

### SUMMARY

Methods are provided for treating a patient suffering from CRC by quantifying the level of a specified p16 fragment peptide in a protein digest prepared from a tumor sample obtained from the patient and calculating the level of p16 peptide in the sample. The peptide may be quantified by selected reaction monitoring using mass spectrometry by comparing the level of the fragment peptide to a defined reference level. The measured levels of the peptide are then compared to corresponding reference levels, and the patient is treated with a therapeutic regimen comprising an effective amount of the combination of cisplatin and pemetrexed therapeutic agents when the level of the p16 fragment peptide is below the reference level. The specified p16 peptide may have the amino acid sequence as set forth as SEQ ID NO:1.

The tumor sample may be a cell, collection of cells, or a solid tissue, such as formalin fixed solid tissue, and/or paraffin embedded tissue. The mode of mass spectrometry may be, for example, tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry and may be carried out using Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

In these methods quantifying the p16 fragment peptide may be quantified by comparing to a spiked internal standard peptide of known amount, where both the native peptide in the biological sample and the internal standard peptide corresponds to the same amino acid sequence of the p16 fragment peptide as shown in SEQ ID NO:1. The internal standard peptide may be an isotopically labeled peptide, such as a peptide labeled with one or more heavy stable isotopes selected from ¹⁸O, ¹⁷O, ¹⁵N, ¹³C, ²H, and a combination thereof.

In the methods described herein the specified level of the p16 peptide fragment may be 108±50 or 108±25 amol/μg protein analyzed.

Detecting and quantitating the specified p16 fragment peptide can be combined with detecting and quantitating other peptides from other proteins in multiplex so that the treatment decision about which agent used for treatment is based upon specific levels of the specified fragment peptide(s) in combination with other peptides/proteins in the biological sample.

### BRIEF DESCRIPTION OF THE DRAWINGS

FIG. 1 shows the relationship between overall survival and microsatellite status.

FIG. 2 shows the relationship between overall survival and tumor mutation burden.

FIG. 3 shows the relationship between overall survival and p16 levels in microsatellite stable CRC patients.

FIG. 4 shows the relationship between overall survival and p16 levels in low tumor mutation burden CRC patients.

### DETAILED DESCRIPTION

Improved methods are provided for treating CRC patients; more specifically the methods involve determining if a CRC patient will clinically respond in a favorable manner to the combination therapy of cisplatin/pemetrexed.

Diagnostic methods are provided for measuring the p16 protein in a tumor sample or samples from the patient. Advantageously the sample is formalin-fixed tissue, particularly formalin fixed paraffin embedded (FFPE) tissue. The amount of the p16 protein in cells derived from FFPE tissue can be determined using an SRM/MRM assay that can measure specific p16 peptide fragments, and particular characteristics about the peptides, at substantially the same time or substantially simultaneously.

A candidate peptide for developing a single SRM/MRM assay for an individual protein may theoretically be any individual peptide that results from complete protease digestion, as for example digestion with trypsin, of the intact full length proteins. Surprisingly, however, many peptides are unsuitable for reliable detection and quantitation of any given protein-indeed, for some proteins no suitable peptide has yet been found. Accordingly, it is impossible to predict which is the most advantageous peptide to assay by SRM/MRM for a given protein, and therefore the specifically-defined assay characteristics about each peptide must be empirically discovered and determined. This is especially true when identifying the best SRM/MRM peptide for analysis in a protein lysate/digest such from FFPE tissue. The present disclosure reports an advantageous peptide for an SRM/MRM assay of p16 from formalin fixed patient tissue. This peptide is also useful in Data Independent Acquisition (DIA) assays for detecting expression with relative quantitation. The peptide sequence for p16 is ALLEAGALPNAPNSYGR (SEQ ID NO:1). Surprisingly it has been found that this peptide can be reliably detected and quantitated substantially at the same time or substantially simultaneously in protein digests prepared from FFPE samples of tumor tissue.

More specifically, this SRM/MRM assay can measure these peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue™ reagents and protocol available from Expression Pathology Inc. (Rockville, Md.). For example, a composition comprising the formalin-fixed tumor sample and a reaction buffer can be heated at a temperature from 80° C. to 100° C. for a period of time from 10 minutes to 4 hours. Additionally, the resulting composition can be treated with an effective amount of a proteolytic enzyme selected from the group consisting of trypsin, chymotrypsin, and endoproteinase Lys-C for a period of time from 30 minutes to 24 hours at a temperature from 37° C. to 65° C. In a particular embodiment, the proteolytic enzyme is trypsin.

The most widely and advantageously available form of tissue, and cancer tissue, from cancer patients is FFPE tissue. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (this is about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from the SRM/MRM assay can be used to correlate accurate and precise quantitative levels of the p16 proteins within the specific cancer of the patient from whom the tissue was collected and preserved, including CRC tissue. This not only provides diagnostic/prognostic information about the cancer, but also permits a physician or other medical professional to determine appropriate therapy for the patient. In this case, utilizing these assays can provide information about specific levels of p16 protein expression substantially simultaneously in cancer tissue and whether or not the patient from whom the cancer tissue was obtained will respond in a favorable way to the combination therapy of cisplatin/pemetrexed.

Treating cancer patients with cisplatin, and most commonly in combination with the drug pemetrexed, is one of the most common and effective strategies for preventing cancer from growing or retarding the growth of cancer cells, and thus prolonging the lives of cancer patients, especially CRC patients.

Presently the most widely-used and applied methodology to determine protein presence in cancer patient tissue, especially FFPE tissue, is immunohistochemistry (IHC). IHC methodology utilizes an antibody to detect the protein of interest. The results of an IHC test are most often interpreted by a pathologist or histotechnologist. This interpretation is subjective and does not provide quantitative data that are predictive of sensitivity to therapeutic agents that target specific oncoprotein targets, such as cisplatin/pemetrexed sensitivity in a p16⁺ tumor cell population.

Research from other IHC assays, such as the Her2 IHC test, suggest the results obtained from such tests may be wrong. This is probably because different labs have different rules for classifying positive and negative IHC status. Each pathologist running the tests also may use different criteria to decide whether the results are positive or negative. In most cases, this happens when the test results are borderline, meaning that the results are neither strongly positive nor strongly negative. In other cases, tissue from one area of cancer tissue can test positive while tissue from a different area of the cancer tests negative. Inaccurate IHC test results may mean that patients diagnosed with cancer do not receive the best possible care. If all or part of a cancer is positive for a specific target oncoprotein but test results classify it as negative, physicians are unlikely to recommend the correct therapeutic treatment, even though the patient could potentially benefit from those agents. If a cancer is oncoprotein target negative but test results classify it as positive, physicians may recommend a specific therapeutic treatment, even though the patient is unlikely to get any benefits and is exposed to the agent's secondary risks.

Thus there is great clinical value in the ability to correctly evaluate quantitative levels of the p16 proteins in tumors, especially CRC tumors, so that the patient will have the greatest chance of receiving the most optimal treatment.

Detection of peptides and determining quantitative levels of specified p16 fragment peptides may be carried out in a mass spectrometer by the SRM/MRM methodology, whereby the SRM/MRM signature chromatographic peak area of each peptide is determined within a complex peptide mixture present in a Liquid Tissue™ lysate (see U.S. Pat. No. 7,473,532, as described above). Quantitative levels of the p16 protein are then measured by the SRM/MRM methodology whereby the SRM/MRM signature chromatographic peak area of an individual specified peptide from each of the p16 proteins in one biological sample is compared to the SRM/MRM signature chromatographic peak area of a known amount of a “spiked” internal standard for the specified p16 fragment peptide. In one embodiment, the internal standard is a synthetic version of the same exact p16 fragment peptide, where the synthetic peptide contains one or more amino acid residues labeled with one or more heavy isotopes. Such isotope labeled internal standards are synthesized so that mass spectrometry analysis generates a predictable and consistent SRM/MRM signature chromatographic peak that is different and distinct from the native p16 peptide chromatographic signature peaks and which can be used as comparator peaks. Thus when the internal standard is spiked in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

In order to develop the SRM/MRM assay for p16 fragment peptides, additional information beyond simply the peptide sequence needs to be utilized by the mass spectrometer. That additional information is important in directing and instructing the mass spectrometer, (e.g., a triple quadrupole mass spectrometer) to perform the correct and focused analysis of the specified p16 fragment peptides. An SRM/MRM assay is that such an assay may be effectively performed on a triple quadrupole mass spectrometer. That type of a mass spectrometer may be considered to be presently the most suitable instrument for analyzing a single isolated target peptide within a very complex protein lysate that may consist of hundreds of thousands to millions of individual peptides from all the proteins contained within a cell. The additional information provides the triple quadrupole mass spectrometer with the correct directives to allow analysis of a single isolated target peptide within a very complex protein lysate that may consist of hundreds of thousands to millions of individual peptides from all the proteins contained within a cell. Although SRM/MRM assays can be developed and performed on any type of mass spectrometer, including a MALDI, ion trap, ion trap/quadrupole hybrid, or triple quadrupole, presently the most advantageous instrument platform for SRM/MRM assay is often considered to be a triple quadrupole instrument platform. The additional information about target peptides in general, and in particular about the specified p16 fragment peptides, may include one or more of the mono isotopic mass of each peptide, its precursor charge state, the precursor m/z value, the m/z transition ions, and the ion type of each transition ion. The peptide sequence of the specified p16 fragment peptide used in the methods described herein is shown in Table 1.

TABLE 1

| SEQ ID NO | Protein | Peptide Sequence |
|---|---|---|
| 1 | p16 | ALLEAGALPNAPNSYGR |

To determine an appropriate reference level for p16 quantitation, tumor samples are obtained from a cohort of patients suffering from cancer, in this case CRC. The CRC tumor samples are formalin-fixed (and optionally paraffin embedded) using standard methods and the level of p16 in the samples is measured using the methods as described above. The tissue samples may also be examined using IHC and FISH using methods that are well known in the art. The patients in the cohort are treated with the combination of cisplatin and pemetrexed therapeutic agents and the response of the patients is measured using methods that are well known in the art, for example by recording the overall survival of the patients at time intervals after treatment. A suitable reference level (e.g., 108 amol/μg total protein) can be determined using statistical methods that are well known in the art, for example by determining the lowest p value of a log rank test. Once a reference level has been determined it can be used to identify those patients whose p16 expression levels indicate that they may likely benefit from the combination of the combination cisplatin/pemetrexed therapeutic regimen. Levels of p16 in patient tumor samples are typically expressed in amol/μg, although other units can be used. The skilled artisan will recognize that a reference level can be expressed as a range around a central value, for example, ±250, 150, 100, 50 or 25 amol/μg.

For those patients where protein expression, as measured using the methods described herein, indicates that treatment with cisplatin plus pemetrexed is unlikely to be effective, an alternative therapeutic regimen may be used. Other therapeutic regimens include surgery (including wedge resection, segmental resection, lobectomy and pneumonectomy), radiation therapy, and targeted drug therapy (such as treatment with Afatinib (Gilotrif), Bevacizumab (Avastin), Ceritinib (Zykadia), Crizotinib (Xalkori), Erlotinib (Tarceva), Nivolumab (Opdivo) and Ramucirumab (Cyramza)).

Because both nucleic acids and protein can be analyzed from the same Liquid Tissue™ biomolecular preparation it is possible to generate additional information about disease diagnosis and drug treatment decisions from the nucleic acids in the same sample upon which the proteins were analyzed. For example, if the p16 proteins are expressed by certain cells at increased levels, when assayed by SRM, the data can provide information about the state of the cells and their potential for uncontrolled growth, choice of optimal therapy, and potential drug resistance. Information, for example, about tumor mutation burden and microsatellite information may also be useful for assessing prognostic outcomes, either considered individually or in combination with data about p16 levels. At the same time, information about the status of genes and/or the nucleic acids and proteins they encode (e.g., mRNA molecules and their expression levels or splice variations) can be obtained from nucleic acids present in the same Liquid Tissue™ biomolecular preparation. Nucleic acids can be assessed simultaneously to the SRM analysis of proteins, including the p16 proteins. In one embodiment, information about the p16 proteins and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

In one embodiment, a method of treating a patient suffering from cancer, especially colorectal cancer (CRC), is provided comprising:
a) quantifying the level of a p16 fragment peptide, for example SEQ ID NO:1, in a protein digest of a tumor tissue sample obtained from the patient, such as a FFPE tissue sample, and calculating the level of the p16 peptides in said sample by selected reaction monitoring using mass spectrometry;
b) comparing the level of said p16 fragment peptide to a p16 reference level, and
c) treating the patient with a therapeutic regimen comprising administration of the combination of cisplatin and pemetrexed or another effective cancer therapeutic regimen when the level of the p16 fragment peptide is below said p16 reference level.

### EXAMPLES

### Example 1

In FFPE archived clinical samples of CRC, 76 proteins were analyzed using mass spectrometry. MSI was measured by whole genome sequencing. Unstable loci were quantified in tumor and normal samples. Cutoffs were derived via receiver operating characteristic (ROC) analysis. High tumor mutation burden (TMB) was defined as >4.5 somatic mutations per megabase of genome analyze. High p16 as ≥108 amol/μg of total protein analyzed. Patients were grouped by microsatellite status (microsatellite instable [MSI] vs. microsatellite stable [MSS]), TMB (high vs. low), and p16 protein expression level. Global proteomic profiling was performed in 30 CRC samples. LC-MS/MS was carried out on an Ultimate 3000 UH PLC system coupled to Q-Exactive HF (Thermo Fisher Scientific). Data Independent Acquisition (DIA) was performed to identify and quantify proteins.

A total of 3757 proteins were identified including known proteins over-expressed in CRC. DIA data analysis was performed using Spectronaut™ Pulsar (Biognosys, Schlieren, Switzerland) software. Normalized DIA readouts of detected proteins were used for unsupervised hierarchical clustering (Ward's method).

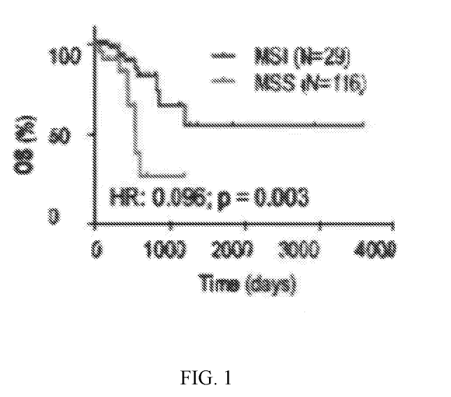

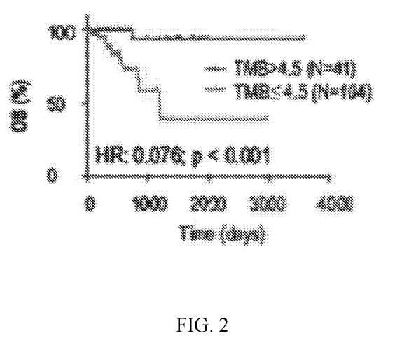

FIG. 1 illustrates that patients with MSI tumors had longer overall survival (OS) than patients with MSS tumors (HR: 0.096; p=0.003). FIG. 2 illustrates that patients with high TMB had longer OS than those with low TMB (HR: 0.076; p<0.001). Quantitative proteomic analysis of CRC is an emerging high-throughput method to collect large amounts of molecular data that linked to tumor phenotype and outcome. The prognostic value of targeted biomarkers in the TMB-low and MSS populations of CRC was assessed. Among the patients with worst outcome, it was found that p16 expression characterized a subset of patients with longer survival.

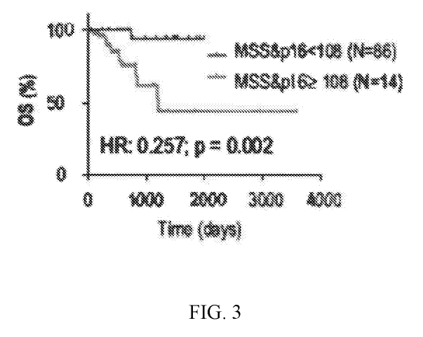

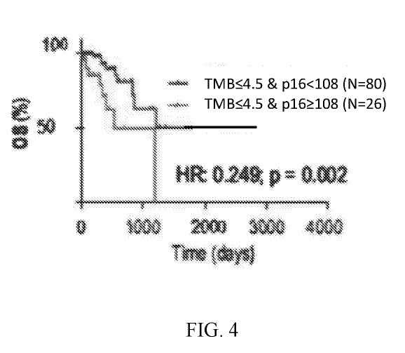

These studies show that 108 amol of p16 peptide per microgram of total protein is a cutoff value that is particularly predictive of overall survival. FIGS. 3 and 4 show that patients exhibiting high p16 protein expression (≥108 amol/μg) had the poorest survival (HR: 2.874; p=0.019) in the all population. Among patients with MSS tumors (FIG. 3) or low TMB (FIG. 4), those with low p16 levels had longer OS than patients with high p16 (HR: 0.257; p=0.002 and HR: 0.249; p=0.002, for MSS and low TMB, respectively).

What is claimed is:

1. A method of treating a patient suffering from colon cancer, the method comprising: administering cisplatin and pemetrexed to the patient,
wherein a protein digest of a formalin-fixed tumor sample from the patient evidences a level of p16 fragment peptide below 108±50 amol/μg protein, and
wherein the p16 fragment peptide is the peptide according to SEQ ID NO:1.

2. The method of claim 1, further comprising heating a composition comprising the formalin-fixed tumor sample and a reaction buffer at a temperature from 80° C. to 100° C. for a period of time from 10 minutes to 4 hours.

3. The method of claim 2, further comprising: treating the resulting composition with an effective amount of a proteolytic enzyme selected from the group consisting of trypsin, chymotrypsin, and endoproteinase Lys-C for a period of time from 30 minutes to 24 hours at a temperature from 37° C. to 65° C.; and assaying p16 fragment peptide level by mass spectrometry.

4. The method of claim 3, wherein the proteolytic enzyme is trypsin.

5. The method of claim 4, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

6. The method of claim 4, wherein a mode of mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

7. The method of claim 1, wherein the tumor sample is a cell, collection of cells, or a solid tissue.

8. The method of claim 7, wherein the tissue is paraffin embedded tissue.

9. The method of claim 6, wherein quantifying the p16 fragment peptide comprises determining the amount of the p16 fragment peptide in said sample by comparing to a spiked internal standard peptide of known amount, wherein both the native peptide in the biological sample and the internal standard peptide correspond to SEQ ID NO:1.

10. The method of claim 9, wherein the internal standard peptide is an isotopically labeled peptide.

11. The method of claim 10, wherein the isotopically labeled internal standard peptide comprises one or more heavy stable isotopes selected from ¹⁸O, ¹⁷O, ¹⁵N, ¹³C, ²H, and a combination thereof.

12. The method of claim 1, wherein the colon cancer is stage III colon cancer.

13. The method of claim 1, wherein the formalin-fixed tumor sample evidences a level of p16 fragment peptide below 108±25 amol/μg protein.


SEQUENCE LISTING (1 sequence)

SEQ ID NO: 1 - protein, 17 aa, Homo sapiens
ALLEAGALPNAPNSYGR